(19) United States
(12) Patent Application Publication
(10) Pub. No.: US 2011/0105969 A1
(43) Pub. Date: May 5, 2011

(54) METHOD AND ORTHOTIC SYSTEM FOR REHABILITATING NEUROLOGICALLY IMPAIRED GAIT

(76) Inventor: Richard A. Nace, San Jose (CR)

(21) Appl. No.: 12/916,597

(22) Filed: Oct. 31, 2010

Related U.S. Application Data
(60) Provisional application No. 61/257,029, filed on Nov. 1, 2009.

(51) Int. Cl. A61F 5/00

(52) U.S. Cl. 602/16; 602/26; 602/27

(57) ABSTRACT

A gait corrective and neural rehabilitative method and orthotic bracing system for use on a patient's lower leg, foot and ankle is provided. The method and system comprises an ankle foot orthosis having a flexible foot plate for positioning below a foot of the patient, an upwardly extending strut connected to the foot plate at a lower end and a cuff system and strap attached the strut for positioning around a shin area of the patient's lower and for securing the ankle foot orthosis to the patient's lower leg. A knee brace is also provided and has at least one upwardly extending strut, at least one hinge member intermediately disposed there along, a means for attaching the knee brace to the patient's lower leg, a means for providing leg swing assistance and a means for increasing patient proprioception.

### PRIOR APPLICATIONS

This non-provisional patent application is a continuation-in-part of U.S. provisional patent application Ser. No. 61/257,029, filed on Nov. 1, 2009.

### FIELD OF THE INVENTION

This invention relates to a method and orthotic system for rehabilitating neurological impaired gait. In particular, the method utilizes and the orthotic system includes a carbon fiber or composite ankle foot orthosis with a knee orthosis having an extension swing assist mechanism, which when used in combination provide foot drop correction, knee support, improved balance, enhanced proprioception, and improved gait biomechanics in patients with significant neurological dysfunction affecting normal gait.

### BACKGROUND OF THE INVENTION

Orthotic devices and appliances commonly referred to as “orthotics,” are known in the prior art and have been utilized for many years by orthotists (a maker and fitter of orthotics), physical therapists and occupational therapists. They are primarily used to protect an injured or surgically repaired ankle or knee joint or a weakened joint caused by neurological disability. They are also used to assist in muscle weakness and to protect the ankle and knee joint as well as to assist in the rehabilitation of a patient's joints and associated limbs or adjacent skeletal parts of the patient's body related to foot drop, ankle and knee instability, quadriceps weakness, loss of proprioception (i.e., perception governed by proprioceptors, as awareness of the position of one's affected leg in space), poor balance and a significant loss in ankle power (inability to push off) and knee power (flexion to extension movement).

Webster's New College Dictionary defines “orthotics” as a branch of mechanical medical science that deals with the support and bracing of weak or ineffective joints or muscles. The word “ortho” comes from Greek and means “to straighten.” Orthotic devices are used to support and straighten an effected joint and assist to correct normal human function as closely as possible. Orthotic devices used as limb braces have typically been designed to support and protect the joint that is associated with ankle or knee injury, for post surgical use and for alleviating pain associated with joint movement at the particular location being treated. There are very few orthotic braces that are designed and utilized to correct abnormal biomechanical movement of a neurologically dysfunctional ankle and/or knee. Further, there are no prior art orthotic braces that are designed to rehabilitate the correct “muscle firing sequence” of the ankle and knee to a more normal gait. Muscle firing sequence is the precise timing of the initiation of muscle contraction and muscle relaxation of the agonist and antagonist muscles of the leg during biomechanically correct gait. Patients who have suffered a traumatic brain injury, stroke or who have Cerebral Palsy, MS or other neurological disabilities can suffer from muscle weakness, loss of proprioception, poor balance, foot drop, loss of ankle and knee power and an altered muscle memory, which can result in abnormal gait and incorrect muscle firing patterns. The gait of a neurologically dysfunctional patient is often significantly altered due to neurological deficits (when the muscle over or under fires,) from muscle weakness and/or abnormal muscle compensation.

Foot drop is a condition wherein the patient experiences a significant decrease or total loss in dorsiflexion (the movement that decreases the angle between the foot and the leg that occurs at the ankle during gait) and is further unable to volitionally lift the foot when attempting to walk due to neurological deficits. The ankle may be flaccid (a condition of reduced muscle tone and/or significant weakness) or affected by abnormal tone or spasticity causing an involuntary plantarflexion (the opposite of dorsiflexion or defined as the movement which increases the approximate 90° angle between the front part of the foot and the shin) or downwards flexing reflex of the ankle/foot with weight bearing, thereby in essence lengthening the effected leg and making normal walking extremely difficult.

To compensate for foot drop, a patient may “hip hike” or lift the effected side hip when walking and swing the effected leg outwards circumferentially to advance the leg forward (also known as “circumduction”). Further, because of a loss in movement of the ankle combined with significant quadriceps weakness, the patient walks with a relatively stiff leg which causes reduced movement at the ankle, reduced hip flexion and reduced knee flexion. When repeated hundreds or thousands of times during recovery from stroke, brain injury, spinal cord injury or other neurological dysfunction, an abnormal muscle firing muscle memory pattern develops, thereby making rehabilitation that much more difficult to correct.

Hemiplegic gait is described as circumduction of the hip, significantly decreased hip and knee flexion, loss of proprioception (knowing where the limb is in space), balance deficits, foot drop and a loss of ankle and knee power. With hemiplegic gait, the patient walks with a straight leg and must lift and circumduct the leg outwardly due to the increased length of the effected leg caused by the foot drop.

Antalgic gait is a neurological condition affecting balance and gait abnormalities and limitations in joint movement characterized by foot drop secondary to tibialis anterior (a muscle that originates in the upper two-thirds of the lateral surface of the tibia and inserts into the medial cuneiform and first metatarsal bones of the foot) weakness resulting in increased hip flexion and knee flexion and circumduction of the entire leg during gait. Assistive devices such as walkers or canes are often required when walking with hemiplegic gait or antalgic gait because of poor balance and a significantly altered gait. Unfortunately, these devices, as well as all other devices in the prior art, do nothing to correct the gait or rehabilitate effected muscles. However, both hemiplegic gait and antalgic gait can be corrected with the present invention, as will be discussed further below hereinafter.

Ankle and knee instability caused by neurological dysfunction is due to an underlying damage to the brain or spinal cord from such injures as a lesion of the brain caused by stroke, MS, Cerebral Palsy, brain tumor, traumatic brain injury and other neurologic insult such as spinal cord injury, which causes weakness in the muscles supporting the ankle and knee joint during locomotion and other activities. Often, separate supportive and protective ankle and knee braces are required to prevent foot drop and knee buckling and secondary damage to the ankle and knee resulting in more serious injury. This is a serious limitation in the prior art, which needs improvement thereupon.

In all of the above listed conditions, one of the most significant conditions is muscle weakness in the upper leg associated with the quadriceps muscles. Knee braces used to treat knee instability and muscle weakness must provide medial, lateral and rotational support of the knee during walking and other activities to adequately support and protect the knee joint to be effective.

Most prior art knee braces are designed to support and protect the knee joint after injury or surgery or to improve knee instability due to weakened muscles. These prior art knee braces are commonly used for knee instability for patients with neurological deficits to provide knee support, but do little to facilitate a normal gait or walking with a normal sequence or for the timing of muscle firing necessary for efficient and balanced ambulation. These prior art devices are considered supportive braces only and are not corrective or rehabilitative. Further, in most cases, the support of the knee brace can actually slow muscle strengthening and neuro-rehabilitation of normal muscle firing because they significantly reduce or eliminate the need to fully use the quadriceps muscles. This further retards weakness in the quadriceps, which is a limiting factor in restoring normal gait biomechanics.

Muscle memory is instrumental to a normal gait and walking kinetics. The firing pattern sequence of each muscle used when walking is stored in the spinal cord in the Central Pattern Generators (CPGs), which allows normal human locomotion to be repeated. When a lesion in the brain or neural disruption affects the neurological system's ability to send the correct signals to the muscles to fire in the precise pattern required for normal ambulation, muscle strength loss and muscle firing patterns are disrupted. As a person recovering from such neurological dysfunction attempts to walk, abnormal muscle firing patterns develop that override or alter normal walking muscle memory. A new abnormal muscle memory is therefore established, which results in alterations in muscle use. This then causes timing errors, which in turn negatively affects the ability to walk normally. As the altered walking pattern is reinforced through repetition, muscle memory in the CPGs becomes altered and can be difficult to restore to a more normal sequence of muscle firing and use. Most prior art orthotics can actually slow rehabilitation by being supportive and protective as opposed to corrective in facilitating more normal biomechanics and enhanced muscle firing patterns.

Foot drop is the direct result of a lack of firing of the tibialis anterior, extensor hallicus longus and extensor digitorum longus muscles. These muscles are responsible for dorsiflexion or lifting of the foot during gait. A corrective and rehabilitative ankle/foot orthosis (AFO) would assist the ankle and foot in making a correctly timed dorsiflexion movement and subsequent plantarflexion movement at toe-off, during gait, resulting in improved firing of the neurologically neglected muscles as well as rehabilitating muscle memory by improving the timing or sequencing of muscle firing. Improved muscle firing can strengthen the weakened muscles of the leg to improve gait over time with repeated brace use. However, before the present invention herein, which will be discussed in greater detail below, no such single device or method of combining known bracing devices exists to rehabilitate ankle/foot and knee biomechanics so that the patient's condition improves independent of the braces employed.

The prior art of ambulating ankle foot orthotics currently available either significantly reduce ankle/foot movement to support the weakened ankle and prevent foot drop through immobilization or by limiting motion to prevent the foot drop and provide ankle support (as seen with solid ankle AFOs and articulating AFOs). While being utilized these AFO orthotics are supportive, but provide no neurological rehabilitation to reverse foot drop with repeated use. With the prior art devices, the ankle muscles can actually lose muscle strength over time because movement of the ankle is restricted or limited. The patient then becomes reliant on the ankle foot orthosis (AFO) for support and is not properly rehabilitated.

Other ankle foot orthotic designs, such as a leaf spring AFO or “dynamic” AFOs provide a dorsi-assist to lift the foot upwards with a spring action of the orthosis to provide dorsiflexion movement of the ankle/foot and to facilitate patient improved gait. Since it is the AFO that is supplying the movement, there is little to no firing of the tibialis anterior, extensor hallicus longus and extensor digitorum longus muscles. These prior art AFOs are corrective in that they provide the improved movement of the ankle to improve gait. However, they too are not rehabilitative because they do not initiate significant muscle firing of the effected muscles, so there is no improvement over time in muscle strength or muscle memory. Again, the patient becomes dependent on the AFO to walk more effectively. These prior art corrective “non-rehabilitative” AFOs also lead to further muscle weakness in the effected ankle, making the patient long term dependent on the device for ambulation.

Most recently, carbon fiber or composite AFOs for foot drop have been introduced in the prior art. These composite AFOs are often referred to as “ground reaction force dynamic AFOs” because they facilitate a heel-to-toe ground reaction movement at the foot, which facilitates heel-to-toe foot placement and movement (AFO footplate action), which in turn loads a strut connected from the footplate to an anterior or posterior cuff providing assistance, which in turn moves the lower leg forward at toe-off (i.e., when the complete foot leaves the ground) during gait to facilitate a more efficient gait.

Carbon fiber or composite AFOs are available with an anterior shell or posterior shell or cuff located below the knee. The struts from the shell to the footplate vary from posterior struts connecting to a posterior shell or medial or lateral curved struts, initiating from the medial or lateral side of the foot with a slight curve from the foot to leg midline, then attaching to an anterior shell and medial or lateral curved strut, initiating from the medial or lateral side of the foot, with a slight curve from the foot to a posterior shell. Other designs include spiral designs, where the strut initiates from the medial or lateral edge of the foot behind mid-foot and wraps around the leg to some degree to attach to either an anterior or posterior shell. Most of these carbon fiber or composite dynamic ground reaction-force AFOs have rigid or semi-rigid foot plates, wherein when weight is placed on the heel, the device moves the ankle quickly to the toe. This ground reaction force initiates ankle/foot movement and a shift from weight balance on the heel of the foot to the toe.

Carbon Fiber or composite ground reaction dynamic AFOs are corrective gait orthotics in that they do initiate corrective movement of the ankle/foot and the extension swing of the lower leg to improve gait mechanics. But, it is the action of the brace that improves the gait. Accordingly, there is little or no improvement in muscle firing of the effected muscles of the ankle as the brace is supplying the movement, not the firing of the patient's muscles. Because of a loss of proprioception, the patient is unable to recognize when to fire the appropriate muscles of the leg when using a carbon fiber AFO. Accordingly, muscle strengthening is not achieved with routine brace use with the prior art devices or any known methods of combination thereof. Improvement of muscle firing sequence or muscle memory is also not achieved; as the effected muscles are not being significantly engaged when walking with these prior art AFOs. However, if combined with a knee brace that overlapped the carbon fiber AFO and provided significant proprioceptive input to the patient's affected leg at the calf and shin, bend in the knee, and at the thigh, the patient would then be able to more significantly benefit from carbon fiber ground reaction force AFO's by knowing when to fire or use their own muscles because they would know when the foot was going to hit the ground. However, this revelation is not known to those practicing in the art of orthotics, and is not taught as a method of rehabilitation for Hemiplegic gait.

Prior art knee bracing is also primarily supportive in nature, intended to control knee movement, improve knee instability and for protecting the knee joint. These prior art knee braces make ambulation possible, aided or unassisted, by supporting the knee joint. However, the neurologically challenged patient will continue to walk abnormally with circumduction of the lower leg, even though confidence is increased with the support provided at the knee with the knee brace. These braces are not designed to correct the gait of a patient with hemiplegic or antalgic gait or patients with other neurologically affected gait patterns.

Quadriceps weakness is a significant problem facing those who desire to rehabilitate a patient with hemiplegic, antalgic gait or other neurologically effected gait patterns. The quadriceps muscles atrophy and lose strength very quickly after a stroke, after traumatic brain injury, in a spinal cord injury or other immobilizing neurological deficits. The abnormal gait adapted by patients with neurological deficits fails to allow the patient to walk with a normal heel-to-toe placement and proper weight shift on the foot during gait. This is due to the inability to affectively extend the leg because of weakness to the quadriceps. The patient lands the foot during gait flat footed, often with the foot pointed outwards with the hip externally rotated (circumduction movement). Heel-to-toe foot placement and weight shift is critical to full engagement of the quadriceps muscles and for initiating neurological firing of such muscles.

Aquatic movement therapy is often used as a method to significantly reduce the weight of the body on the legs to enhance the patient's ability to correctly move the legs more easily. Walking in the water is much easier than on land, as the load on the limbs is significantly reduced due to weight displacement in the water, balance is thereby enhanced and a more normal movement pattern of the legs can be realized to provide effective muscle strengthening exercise as well as improving neuro rehabilitation with more normal leg movement. Constructing orthotic braces utilizing a similar concept of therapy, whereby the work required for the muscles is reduced while facilitating active volitional muscle firing with enhanced muscle firing sequencing has significant rehabilitative benefits not found in existing orthotic braces. Assisting corrective movement, facilitating significant proprioceptive input below, at, and above the knee, while requiring some volitional firing of the effected muscles to walk with a normal gait, can lead to significant gains in strength and muscle memory over time. This approach to bracing is more effective, as the patient does not become brace dependent for increased mobility or ambulation, but instead uses the rehabilitative braces to strengthen the effected muscles and to improve walking effectiveness so that the patient is actually receiving rehabilitation therapy by the braces as they walk. As muscle strength and muscle timing improves, the patient can transition to less bracing and assistive device support needed to walk over time. Unfortunately, until the present invention, no such brace or method of bracing is known in the prior art.

Numerous clinical studies on neuro-rehabilitation have demonstrated that with massed practice a patient recovering from a stroke or other neurological deficit can re-learn movement patterns and regain permanent functional use of effected limbs. Thousands of repetitions of purposeful movement are required to rehabilitate a patient who has suffered a significant upper motor neuron injury. Most importantly, these studies reinforce that biomechanically corrected movements, if repeated, can provide for meaningful rehabilitation and permanent recovery. Therefore there is a need for a brace, bracing system or method for rehabilitation, which until the present invention is unknown, that satisfies all of the aforementioned deficiencies of the prior art.

Taub's Constraint Induced Therapy clinical studies, as well as Levine, et al.'s (Drake Rehabilitation Center) clinical studies using Modified Constraint Induced Therapy demonstrate that movement patterns can be relearned after thousands of repeated correctly sequenced “practices” until more normal movement patterns are re-learned permanently. Exercises that reinforce correct biomechanical movement necessary for specific task completion must be completed by the therapy participant thousands of times to permanently re-learn correct functional use of the affected limb. Orthotic braces that correct dysfunctional movement and facilitate more normal movements and muscle firing patterns can be used like manually assisted physical or occupational therapy to rehabilitate a limb similar to assisted therapeutic exercise. However, as set froth above, no such brace, bracing system of method for rehabilitation using braces is known in the prior art, until the advent of the present invention.

The prior art does include a bevy of various knee brace designs. Most knee instability brace designs include a rigid anterior thigh cuff, a rigid anterior shin cuff, a rigid medial upright with a unicentric or polycentric hinge, a lateral upright with a unicentric or polycentric hinge, a strapping system, and condyle pads or another stabilizing force system at mid knee joint on both sides, as can be seen in the braces manufactured by the companies Bledsoe™, Ossur™ and Townsend™.

Other knee instability or ligament braces have a rigid anterior thigh cuff, a rigid posterior calf cuff, rigid medial and lateral uprights, condlye pads, a strapping system, and unicentric or polycentric hinges, such as with the Donjoy Defiance™ brace. Still other knee instability knee braces have a rigid posterior thigh cuff, a rigid anterior shin cuff, rigid medial and lateral uprights with polycentric hinges, condyle pads, and a strapping system, such as seen with the Donjoy 4Titude™ brace. The knee joint hinges may or may not have range of motion stops to limit the extension and flexion of the knee within a protected range.

Still other knee instability braces are fabricated from a neoprene type elastic material with one or two rigid unicentric or polycentric hinges (medial or lateral hinge placement) and a patellar reinforced opening at the knee cap to facilitate knee brace alignment and to prevent brace migration. These braces allow the patient to pull on the brace, facilitating donning and doffing for hemiplegic patients and other neurologically compromised patients.

Almost all prior art knee laxity braces lack a dynamic swing assist mechanism to correct abnormal gait associated with a neurologically dysfunctional condition (i.e., hemiplegia). This is a serious disadvantage in the prior art and something that clearly needs to be improved upon. All prior art non-elastic knee laxity braces are also primarily rigid, and do not provide enough proprioceptive stimulation when used on a patient to allow a neurologically dysfunctional patient with hemiplegic gait to significantly improve proprioception when walking in the knee brace.

What is therefore needed is a complete neurological gait training combination of orthotic braces, which includes a flexible carbon fiber or composite ground reaction force dynamic AFO and a knee laxity brace with a dynamic extension swing assist mechanism that can support the knee medially, laterally and rotationally, provide a swing-assist function for extension, provide a corrective and therapeutic force that can return the patient to a more true normal gait (heel-to-toe strike while walking), prevent abnormal rotation of the knee joint, significantly improve leg proprioception with flexible bands of compression above, at, and below the knee joint all the while recruiting atrophied muscles to work again and to rehabilitate themselves so that the patient can once again return to the closest possible “normal” condition based upon the specific progression of their respective condition. The goal for any advancement in the prior art should be an improvement from “abnormal gait” to a more biomechanically correct normal gait kinetic, which is believed to be a significant factor in reducing the rehabilitation necessary to allow for normal ambulation. A truly rehabilitative AFO/knee brace combination system for neurologically dysfunctional gait rehabilitation would strengthen the leg musculature over time using dynamic adjustable components such that the gait, unassisted by the orthotics, improves over time. Such a described brace combination system or method currently does not exist in anywhere in the prior art.

### SUMMARY OF THE INVENTION

The present invention provides a method of using and a system including a carbon fiber or composite ground reaction force dynamic AFO with a supportive knee orthosis, having an extension swing assist mechanism, worn simultaneously, to correct abnormal gait and to rehabilitate musculature above, below and at the knee joint area. Both the AFO and knee brace of the present method and system are fabricated in a wide range of sizes, for either the left or right ankle and foot and knee, to rehabilitate abnormal gait associated with neurological dysfunction, including hemiplegic and antalgic gait. Such a novel combination of braces of the present invention provides easily managed controls for directing the movement of the ankle/foot and knee directly and the hip indirectly, such that no bracing of the hip is needed. The AFO used in the present invention assists in corrective movement of the ankle/foot while encouraging muscle activation by the patient during gait. The knee orthosis used in the present invention provides medial, lateral, and rotational support of the knee during ambulation and other activities, as well as correcting abnormal gait and facilitating heel-to-toe walking using a dynamic extension swing assist mechanism to improve gait and to increase the firing of the quadriceps muscles. The dynamic extension swing assist mechanism can be located on either the lateral or medial side of the knee brace and is mounted thereupon in the preferred embodiment. However, in alternate embodiments, the dynamic extension swing assist mechanism can be an add-on mechanism separate from the knee brace.

The AFO/knee brace method and system combination of the present encourages dorsiflexion at the ankle/foot, knee flexion, hip flexion and improved lower leg extension at toe-off as well improved firing of the quadriceps muscles through facilitated heel-to-toe gait, which in turn leads to the quadriceps muscles strengthening. Still further, the AFO/knee brace method and system combination improves muscle firing sequencing through the gait corrective function.

The system of the present invention incorporated into the knee brace uses a combination of air bladders, elastic, static and/or semi-static straps and flexible and/or rigid cuffs, which generates significant adjustable proprioceptive input by compression, which then allows the neurologically impaired patient to overcome proprioceptive losses and thereby “re-learn” normal walking biomechanics. By increasing proprioceptive stimulation by compression on the effected leg, the patient is more affectively able to volitionally control the movement of the effected leg, thereby significantly improving the patient's ability to coordinate leg movement. Dynamically conformable thigh and shin cuffs with a combination of adjustable elastic, static and semi-static strapping, combined with an adjustable air bladder knee support system on the knee brace, significantly improves patient proprioception input with brace use, which is essential for improved patient control of the effected leg, leading to neuro-rehabilitation of more biomechanically correct gait over time. It should also be noted that all of the air bladders and strapping of the present system and method are adjustable for providing additional therapist or patient control over compression.

By initiating a more normal firing of the quadriceps muscles and correcting abnormal gait, quadriceps muscle strength improves over time as well as gait muscle memory. The present invention, in one embodiment, achieves this significant improvement with an adjustable dynamic fulcrum, positioned on the medial or lateral side of the brace, to allow the clinician to quickly and easily adjust the brace to provide the necessary gait swing assist to facilitate a more normal gait of the overall knee as needed during the gait rehabilitation process.

In a preferred embodiment, the knee instability brace of the present invention provides a system for applying a corrective and therapeutic force to the knee joint area to assist in the traditional functions of a knee instability brace but with the added benefit of actually correcting gait kinetics and providing necessary proprioceptive input to the affected leg to improve volitional control of the leg with gait. Therefore, with such a system, a patient with hemiplegic or antalgic gait will realize a significant reduction or elimination of hip circumduction, a significant support medially, laterally and rotationally at the knee joint area, a transformation from a flat foot placement walk to a near normal heel-to-toe foot placement and a lengthening of their actual step.

The present invention combines any dynamic AFO, such as a carbon fiber or composite ground reaction force dynamic AFO, with a supportive knee brace having an adjustable dynamic swing assist mechanism, which is uniquely designed to provide the corrective gait, foot drop correction, knee support and protection, proprioceptive input and improved knee balance control needed for patients who suffer from neurological deficits in the lower extremity. Knee instability is prevalent in many of these patients who develop significant abnormalities from a normal gait due to the neurological deficits, muscle weakness and knee instability. In such cases, muscle memory needed for normal gait has been disrupted and a gait corrective and muscle rehabilitative knee brace in addition to a supportive brace is needed for rehabilitation of these patients, such as seen with the present invention. There are no knee braces in the prior art that are designed to provide significant proprioceptive stimulation to the affected leg to increase the patient's ability to more effectively control movement of the leg. Carbon fiber or composite AFO's without the proprioceptive stimulation below, at, and above the knee, like provided by the present invention knee brace, do not allow for significant improvements in neuro-rehabilitation of the affected ankle/foot because the patient does not have the ability to know when to fire the appropriate muscles due to proprioceptive deficits.

In one embodiment, a carbon fiber or composite ankle foot orthosis (AFO) has a flexible foot plate for providing a corrective and rehabilitative dorsiflexion assist, an anterior, posterior or spiral strut for providing a dynamic extension swing assist and a cuff at the shin or calf. The AFO employed in the present invention is used in conjunction with a knee orthosis providing medial, lateral, and rotational support of the knee with a dynamic extension swing assist and an adjustable strapping system and air bladders designed to initiate adjustable proprioceptive stimulation in the affected leg. In combination, the effect on the leg is the stabilization of a weakened ankle and knee joint and the ability to more precisely use the affected leg muscles to rehabilitate the leg. It also corrects Hemiplegic, Cerebral Palsy, MS or other neurologically impaired gait by applying adjustable corrective and therapeutic forces all the while stimulating the firing of the affected muscles. Over time, this novel combined brace use incrementally corrects abnormal gait, thereby rehabilitating effected muscles and improving the ability to walk unaided. This is accomplished by increasing proprioceptive stimulation in the affected leg, increasing muscle firing and thereby strengthening the ankle and knee joint and surrounding muscles above and below the knee, which then improves muscle memory such that a more normal gait pattern is realized by recruiting said muscles with brace use in a pattern that more closely approximates normal gait to accelerate neuro-rehabilitation. By providing muscle firing in a corrected gait with device use, muscle strengthening and muscle sequencing neuro-rehabilitation improves walking ability over time without brace use.

Rehabilitative ankle and knee bracing that corrects abnormal gait by facilitating both the firing of underutilized muscles and a more normal sequence of muscle firing as well as significantly improved proprioception is the basis for the present invention. In addition, corrective gait orthotics, by stimulating appropriate muscle firing and sequencing that leads to muscle strengthening is also achieved with the present invention.

Further, the present invention combines the use of a rehabilitative carbon fiber or composite ground reaction force dynamic AFO as described with a supportive and corrective knee orthosis with a swing assist mechanism that facilitates heel-to-toe walking when using the brace. By incorporating a dynamic swing assist mechanism in the knee brace, as the one used in the present invention, to swing the foot forward, it improves or corrects heel-to-toe foot placement. Further, while weight shift occurs, firing of the quadriceps is facilitated. Over time, with routine brace use of the present invention, continual firing of the quadriceps leads to their strengthening. Also, improved gait biomechanics and rehabilitation of more normal muscle memory and gait patterns is also achieved. As the leg becomes incrementally stronger, the gait continues to improve with greater step length, improved leg extension, increased knee power and a more rehabilitated gait. With repeated and routine use, after some time, depending on each particular case and each patient, the patient will realize more leg strength and improved gait without the use of the orthotics devices.

The present invention thus provides a method and system that combines a more flexible carbon fiber or composite ground reaction force dynamic AFO to correct foot drop and to assist in lower leg extension with a supportive knee orthosis with an extension swing assist mechanism to correct knee instability and an adjustable strapping system and air bladder support system to improve proprioception and balance, which facilitates heel-to-toe gait. These work in conjunction (AFO and knee brace) to assist in facilitating correct muscle movement and muscle firing sequencing while at the same time requiring that volitional movement of the effected joints occurs.

More specifically, the present invention is a method and system of combining two separate orthotic devices, a carbon fiber or composite ground reaction force AFO with a knee orthosis having a dynamic extension swing assist mechanism and flexible support bands for providing improved proprioception. Orthotists skilled in fabricating orthotic devices often use a Knee/Ankle/Foot Orthotic or KAFO when support of the ankle/foot and knee is required. However, to obtain the dynamic interface between the ankle/foot device (AFO) and the knee brace and to optimally correct gait in a neurologically impaired patient, the AFO and the knee brace should not be attached, such as in the system and method of the present invention. Therefore, the present invention that incorporates both the method and system is a huge improvement over any known KAFO.

Carbon fiber or ground reaction force dynamic AFOs as described in the present invention have dynamic properties and must be able to move slightly on the leg with gait. Fixing the AFO to a knee orthosis places unwanted forces on the AFO strut, which can lead to AFO breakage. Further, distributed corrective forces initiated by the brace are altered when the AFO is attached to the knee brace. The upper portion of the carbon fiber or composite ground reaction force dynamic AFO must be allowed to “piston” or move slightly up and down within the lower shin or cuff band of the knee brace. In a preferred embodiment of the present invention, the AFO should be applied first and then thereafter the knee brace applied over the AFO. The knee brace should not place any downward forces on the AFO as well, in order to maintain the correct dynamic action of the AFO. The knee brace used in the present method and system has a complimentary interface at the lower band of the knee brace to allow for free movement of both the AFO and the knee brace independent of one another even though the knee brace will overlap a distal end of the AFO. During gait, the AFO will be able to move freely within the knee brace (up and down), and the knee brace movement and function will not be affected by having the distal end of the AFO positioned within the knee brace. This is further accomplished by having suitable interface materials that allow the outside of the AFO and the inside of the knee brace to accommodate free movement of each orthotic with minimal frictional effects.

The present invention utilizes any dynamic AFO of similar design (anterior or posterior shell or cuff design; posterior, medial or lateral placement of the strut to the footplate, and a posterior straight, curved or spiral strut design). In a preferred embodiment, a carbon fiber or a composite ground reaction force dynamic AFO is used. The unique properties of the carbon fiber or composite ground reaction force dynamic AFO is in the fabrication of the AFO so that: (1) the AFO footplate is flexible to encourage a corrective assistive heel-to-toe placement with gait, a weight shift assist from heel-to-toe, and a dorsiflexion movement; all facilitated movement provides some, but not all, of the muscle action necessary for normal gait; muscle firing of the effected muscles is initiated by the brace and additional volitional muscle firing is required to step correctly; (2) the AFO foot plate is more flexible distal to the ankle than proximal to the ankle, initiating an assistive dynamic force to encourage knee flexion assist by creating a “lift” force resulting in a slight lifting of the entire lower leg; (3) a flexible AFO footplate being incrementally more flexible at the toe than the heel to increase ankle power or forward movement of the lower leg at the ankle during toe-off; (4) an AFO strut from the footplate to the anterior or posterior cuff that is more flexible than existing designs and is constructed to facilitate an assisted corrective forward extension swing motion of the lower leg, while not propelling the lower leg forward enough to complete a normal step, requiring additional firing of the leg muscles to complete normal step length; (5) a flexible curved junction of the flexible foot plate and lower strut curvature of the carbon fiber or composite ground reaction force dynamic AFO providing a corrective force to align the ankle foot during the swing phase of gait such that the hip will not rotate externally; and (6) a flexible curved strut that will provide a dynamic corrective force to facilitate correct biomechanical placement of the heel-to-toe at heel strike so that the foot placement is relatively pointed straight ahead and the toe does not migrate externally (toe out) with gait.

The design of the new combination of carbon fiber or composite ground reaction force dynamic ankle foot orthosis device and a single or double upright knee brace design with a dynamic extension swing assist mechanism optimally corrects abnormal gait and facilitates correct heel-to-toe placement when walking in the brace. Gait correction is provided at the ankle/foot, knee and indirectly to the hip when the products are used in combination. Adjustable proprioceptive stimulation provided by a system of elastic and static straps above and below the knee used in combination with adjustable air bladders at the knee joint can provide the proprioceptive input necessary for a patient to know where the affected leg is in space during gait to facilitate improved volitional firing of the leg muscles that would be absent or severely diminished without the proprioceptive stimulation. Effected leg muscles are encouraged to fire with improved proprioceptive stimulation to the affected leg and device corrected assisted movement, leading to muscle strengthening and improved muscle memory to rehabilitate gait with routine combined brace use. Normal proprioception, so critical to gait training, and a more normal gait is significantly improved by the increased proprioceptive stimulation and corrective forces provided by the devices, which is not found in any prior art device system or method.

The design of the present knee brace addresses the importance of maintaining muscle firing of all muscles used during normal gait. The dynamic swing assist mechanism incorporated into the present invention helps to maintain a more normal gait. Heel strike with gait is important in that it is necessary to fire all of the quadriceps muscles. The adjustable dynamic swing assist mechanism is designed to facilitate heel strike during ambulation when wearing the brace. The flexible leg support bands significantly improve proprioception allowing the patient to make significant and permanent improvements in gait over time with product use. Finally, dynamic conformable elements (i.e., flexible shin and thigh cuffs and shin and thigh area strapping) also contribute to the improvement over any prior art device as proper compression is constantly maintained on the knee above and below the knee joint of the leg being treated to allow the patient the proprioceptive input necessary to better control the movement of the affected leg.

### BRIEF DESCRIPTION OF THE DRAWINGS

The detailed description of the invention, contained herein below, may be better understood when accompanied by a brief description of the drawings, wherein:

FIG. 1 is a right side elevational view of an ankle/foot orthosis (or “AFO”) employed on a right leg of person's body by itself;

FIG. 2 is a perspective view of the same AFO shown in FIG. 1;

FIG. 3 is a front elevational view of the same AFO shown in FIGS. 1 and 2;

FIG. 4 is a right side elevational view of an AFO and a knee brace of the present invention employed on a right leg of person's body;

FIG. 5 is a perspective view of the same AFO and knee brace shown in FIG. 4;

FIG. 6 is a front elevational view of the same AFO and knee brace shown in FIGS. 4 and 5; and

FIG. 7 is a right side elevational view of the same AFO and knee brace shown in FIGS. 4-6, wherein the leg of the person wearing the AFO and knee is in a flexed state.

### DETAILED DESCRIPTION OF THE PREFERRED EMBODIMENT

Throughout the following detailed description, the same reference numerals refer to the same elements in all figures.

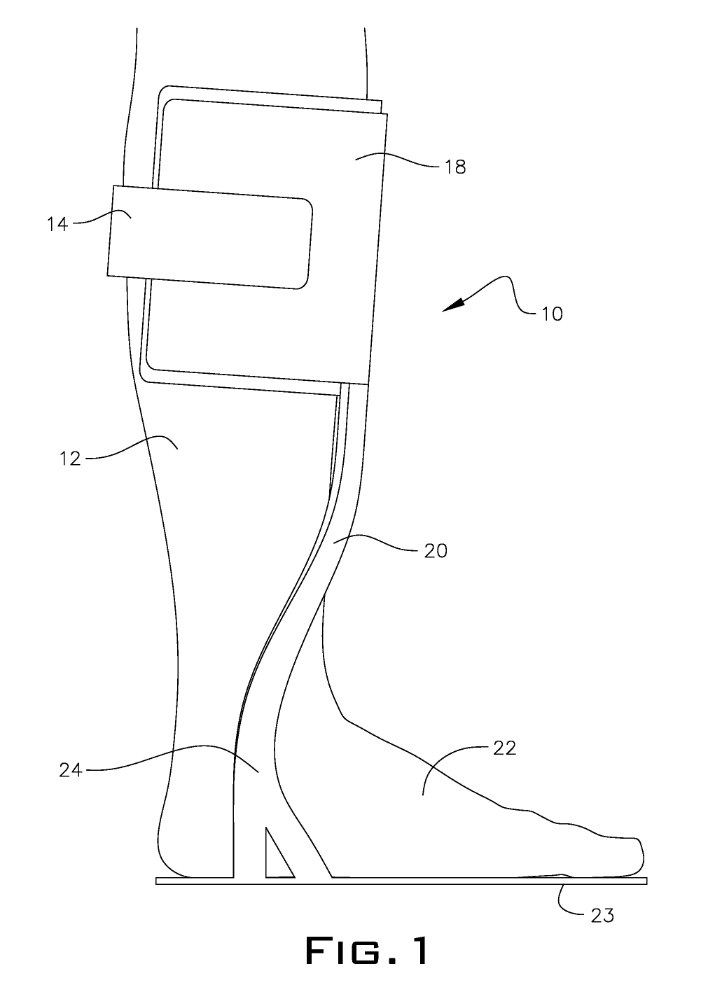

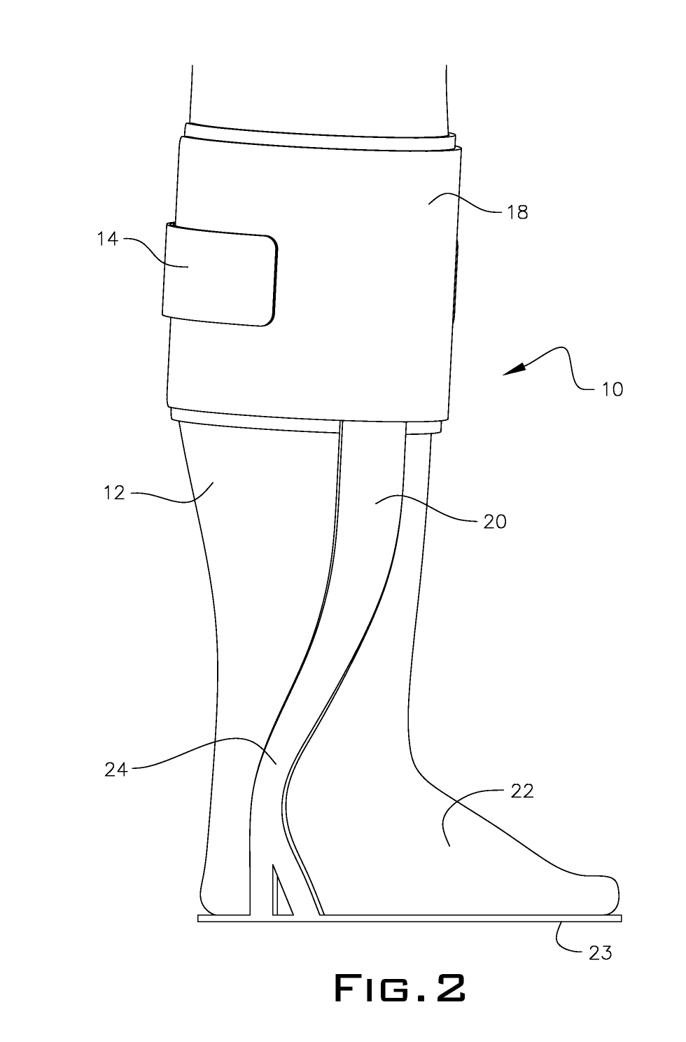

Referring to FIG. 1, an ankle/foot orthosis (AFO) 10 is shown that is employed to a lower leg portion 12 of a patient, which can be used with the method and system of the present invention. Many different types of AFOs can be employed with the novel bracing method and system of the present invention and it is understood that the novel bracing method and system of the present invention is not limited to an AFO of the type shown in FIG. 1 (or FIGS. 2-7, for that matter). Notwithstanding, as shown in FIG. 1, AFO has a single strap 14 that is attached to a front plate 18, which in turn is attached to an upwardly disposed strut member 20. A bottom end of AFO strut member 20 terminates and attaches to a foot plate 23 that inserts within a shoe (not shown) thereby positioning underneath a foot 22, as shown in all FIGS. 1-7. However, as stated just above, other types of AFOS can be used in the novel bracing method and system of the present invention and includes AFOS such as that seen in U.S. Design Pat. No. 583,062, issued Dec. 16, 2008 to Richard A. Nace, which is incorporated by reference herein, and is the preferred AFO to employ.

Other AFOs that can be employed with the present bracing method and system do not require just one strap but instead employ a pair of straps. Further, nothing here requires that strut member 20 be spiral-shaped as in the preferred embodiment, but instead struts that are generally straight-like can be employed. Still further, the AFO can be either a single or a double strut design.

However, in the preferred embodiment, the AFO employed with the novel bracing method and system of the present invention is either a fiber or composite ground reaction force dynamic ankle foot orthosis device having a single strut, a rigid shin cuff having one strap with a single spiral strut as seen in FIG. 1-7.

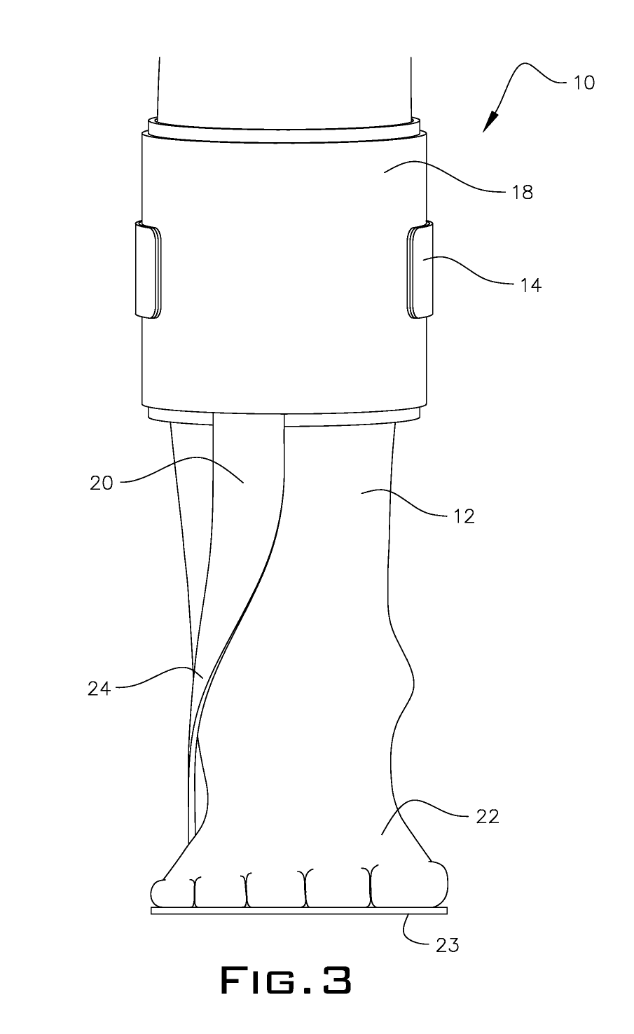

Referring to FIG. 2, the same AFO 10 as shown in FIG. 1 is illustrated and it can be seen that strut member 20 turns slightly to the lateral side of the foot at a lower portion 24. In this particular configuration, it turns slightly to the right because AFO is employed on the right lower leg of the patient. However, if this AFO was employed on the left lower leg, then lower portion 24, turning slightly to the lateral side, would be bending slightly left. It is therefore clearly understood that the novel bracing method and system of the present invention can be used to treat either the right or left leg of a patient by merely employing the proper left or right AFO device. Referring to FIG. 3, the same AFO 10 that is shown in FIGS. 1 and 2 is shown therein but from a front elevational view.

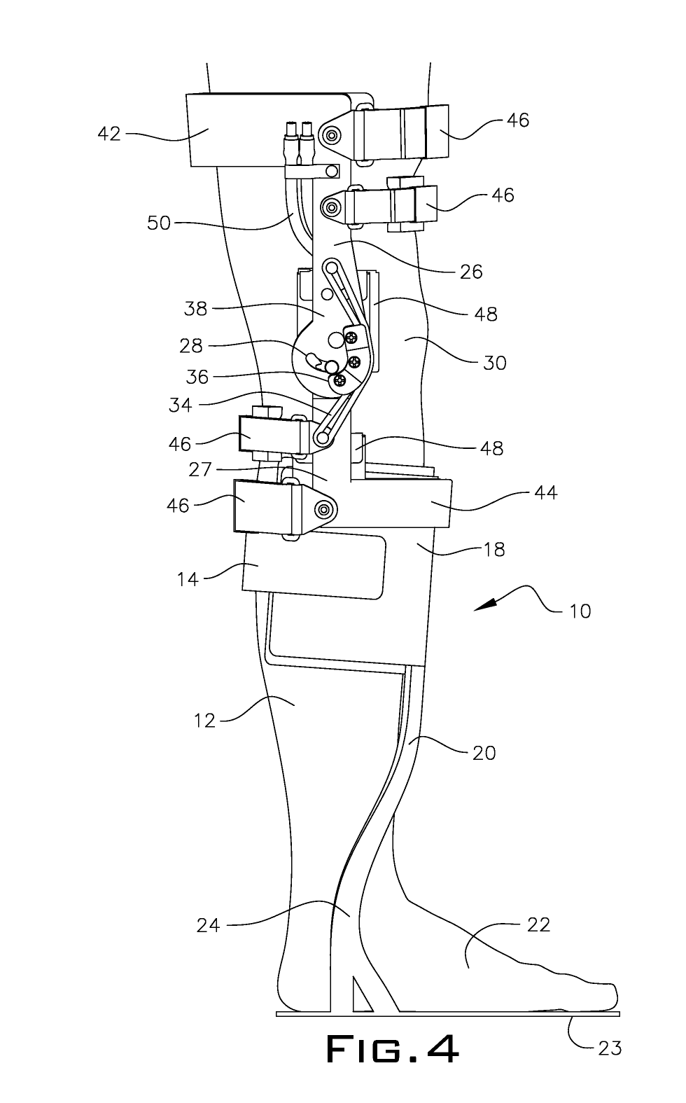

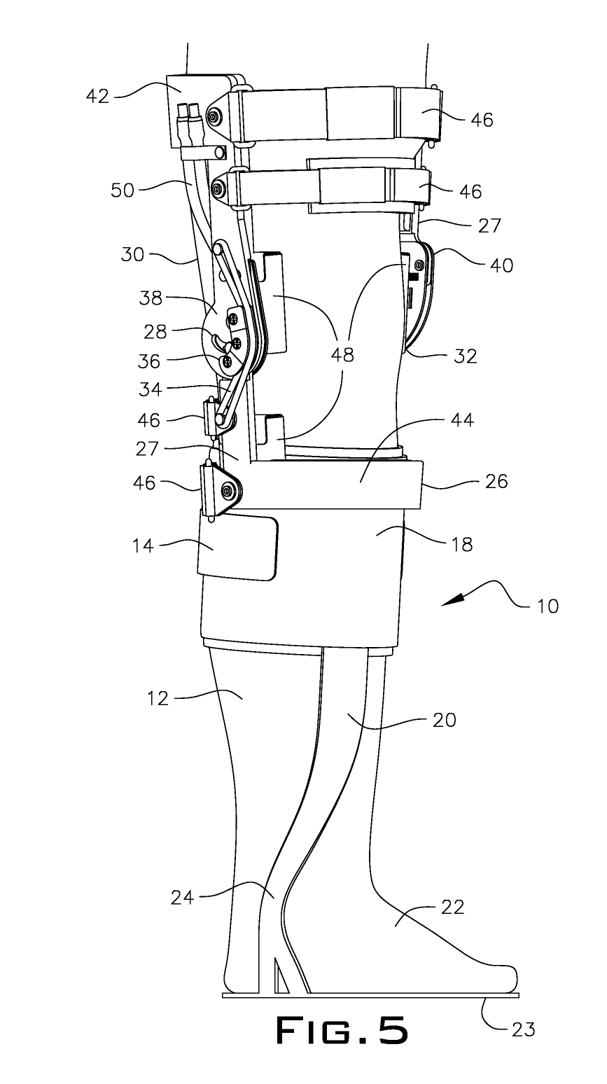

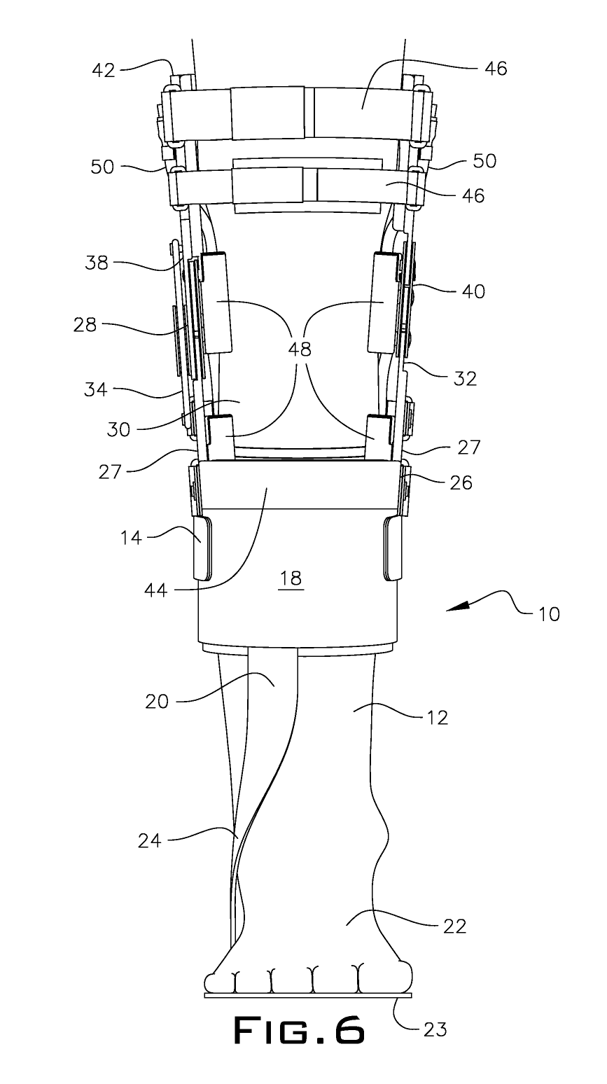

Referring now to FIG. 4 it is shown that in combination with AFO 10, a knee brace 26 is employed around the patient's knee and over AFO 10. Knee brace 26 has a pair of double upright struts 27 (see FIG. 6), although nothing here limits the use of a single upright strut knee brace. Knee brace 26 also employs a dynamic gait swing assist mechanism 28, which in FIGS. 4-7 is shown to be on a lateral side 30 of knee brace 26. Again however, nothing herein limits that dynamic gait swing assist mechanism 28 be solely located on knee brace lateral side 30 and therefore can be located on a medial side 32 (see FIGS. 5 and 6) of knee brace 26. In the preferred embodiment, dynamic swing assist mechanism 28 is an adjustable dynamic fulcrum of which is fully described in U.S. Pat. No. 7,608,051, issue date Oct. 27, 2009, as well as divisional U.S. patent application Ser. No. 12/366,925, filed on Feb. 6, 2009, currently pending, both to Richard A. Nace, and of which both are fully incorporated herein by reference.

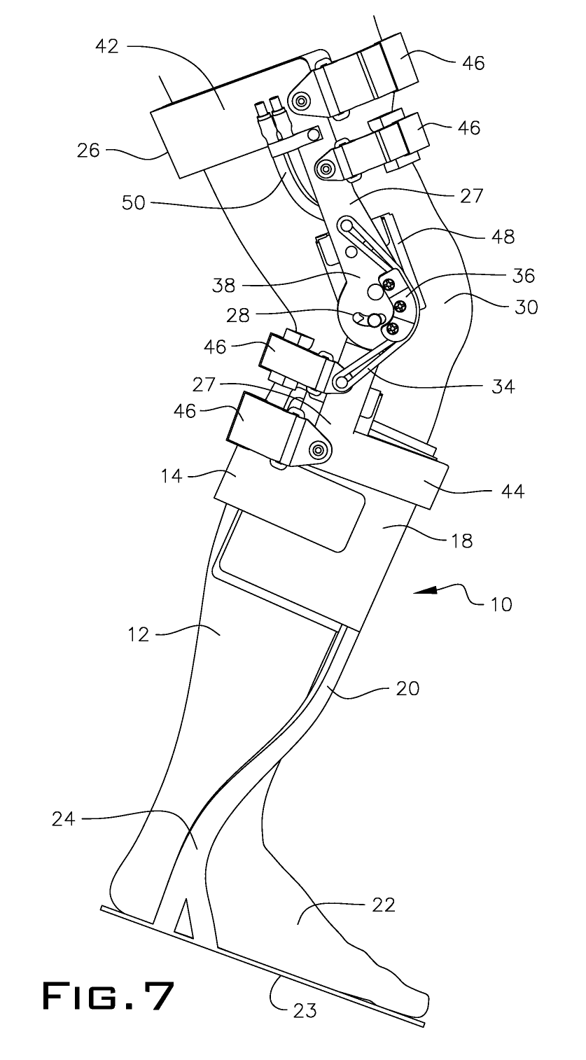

By comparing FIG. 4 versus FIG. 7, one can clearly see how dynamic gait swing assist mechanism 28 is in a non-tensioned state (FIG. 4) when the leg is extended and in a tensioned state (FIG. 7) when the leg is flexed. The tension in the preferred embodiment of dynamic gait swing assist mechanism 28 is provided by an elastic band 34 as shown in FIG. 7, which acts to assist in swinging the leg forward when going from flexion to extension of the leg. Also to the preferred embodiment, elastic band 34 is stretched over one or more adjustable setting blocks 36 (in FIGS. 4, 5 and 7), three blocks 36 are shown. However, although not shown, elastic band 34 can reside within a groove (also not shown) along an outer edge of dynamic gait swing assist mechanism 28.

It is noted and clearly understood that the dynamic gait swing assist mechanism 28 shown and described herein is a preferred embodiment. However, multiple alternate embodiments exist and nothing herein limits their use with the novel method and system for rehabilitating neurological impaired gait. Such alternate embodiments employ springs and/or other known devices, employed alone or in combination, which have the property and/or ability to be in a resting or non-tensioned state and wherein when tensioned have the desire to return to resting or non-tensioned state.

Referring to FIGS. 5 and 6, knee brace 26 has a pair of hinges 38 and 40. Hinge 38 is a polycentric hinge and hinge 40 is a unicentric hinge. Further, as shown in FIGS. 5 and 6, polycentric hinge 38 is disposed along lateral side 30 and unicentric hinge 40 is disposed along medial side 32. However, nothing herein limits the present invention to a method and system for rehabilitating neurological impaired gait to just this one configuration and embodiment. The placement of the polycentric and unicentric hinges, 38 and 40, respectively, can be reversed such that polycentric hinge 38 is disposed on medial side 32 and unicentric hinge 40 is disposed on lateral side 30. Further the method and system for rehabilitating neurological impaired gait of the present invention can be used on either the left or right leg or both legs simultaneously, although FIGS. 1-7 merely show a right leg configuration. Still further, the knee brace employed in the present invention of a method and system for rehabilitating neurological impaired gait can employ a double or single strut design. If it is a single strut design, then the hinge can be either polycentric or unicentric. Still even further, in a double strut design, both hinges can be polycentric or both hinges can be unicentric. Yet still even further, regardless if one or two unicentric hinges are employed, the preferred unicentric hinge, as seen in FIG. 6, has flexion and extension stop settings, such as that seen in U.S. Pat. No. 6,039,709, issued on Mar. 21, 2000 to Bzoch, of which is incorporated by reference herein. Notwithstanding, alternate embodiments are employed for unicentric hinge 40, which do not have the exact mechanism and workings of the above described preferred unicentric hinge.

Referring now to FIGS. 4-7, in a preferred embodiment, it is shown the knee brace 26 is a double upright strut design, although as previously stated, single upright strut-type braces can be employed with the novel method and system of for rehabilitating neurological impaired gait in a patient inflicted with abnormal gait. Further, a posteriorly positioned thigh cuff 42 made of a semi-flexible material, such as Boltaron™ and anteriorly positioned shin cuff 44 of the same or similar semi-flexible material are employed. However, nothing herein limits that knee brace 26 be configured such as this preferred embodiment. As such, thigh cuff 42 can be anteriorly positioned and shin cuff 44 can be posteriorly positioned. Or thigh and shin cuffs, 42 and 44, can both be positioned anteriorly or they both can be positioned anteriorly. Still further, both the thigh and shin cuffs, 42 and 44, respectively, can be made of a more rigid material than that of the preferred embodiment.

Returning to the preferred knee brace 26, used in the novel method and system for rehabilitating neurological impaired gait, strapping 46 is used at the thigh cuff 42 and directly there below and also with the shin cuff 44 there at and directly there above, as clearly shown in FIGS. 4-7. Still further, a unique air bladder system 48, inflated and deflated as required by inflation tubes 50, is employed in the preferred embodiment the knee brace 26 which is fully described in U.S. patent application Ser. No. 12/200,394, filed Aug. 8, 2008, currently pending to Richard A. Nace, of which is fully incorporated herein by reference. Such incorporated reference also discusses the importance of “dynamic conformability,” which knee brace 26 possesses and is part of the preferred embodiment of this novel and non-obvious method and system for rehabilitating neurological impaired gait in a patient inflicted with abnormal gait. It is also noted that the shorten profile (i.e., the height from shin to thigh) of the knee brace used in the preferred method and system of the present invention eliminates many of the problems seen in the prior art as to slippage (i.e., migration) of the knee brace. This is an important improvement over the prior art.

Notwithstanding the preferred embodiment, knee brace 26 can be employed without the use of the air bladder system 48 of the preferred embodiment. Further, there is no requirement that strapping 46, as described above, be employed and therefore a multitude of varying strapping configurations can used with knee brace 26 in the present invention for a method of and system for rehabilitating neurological impaired gait in a patient inflicted with abnormal gait.

In practicing the method of the present invention, in the preferred method, an AFO 10, as previously described above, is affixed to the lower leg of the patient inflicted with abnormal gait first. Thereafter, a knee brace 26, as also described herein above, is then attached over AFO 10 for the reasons as described in the Summary of the Invention above. However, in an alternate embodiment, the affixation is reversed whereby the a knee brace 26, as described above, is first affixed to the lower leg of the patient with the AFO 10 then applied over the knee brace 26.

It should also be noted that FIGS. 1-7 illustrate the preferred embodiment for the system and method of the present invention. However, certain alternate embodiments include the thigh cuff of the knee brace being anteriorly disposed, while the shin cuff is posteriorly disposed. Or both the thigh and shin cuff and posteriorly disposed or both are anteriorly disposed. Further, the thigh and shin cuffs can be rigid, semi-rigid or flexible, or then can be employed in any variation or combination thereof. Still further, the knee brace can be offset at an upper end at the thigh cuff in the height on one side as well as the posterior positioning or angle of the thigh cuff. In an off-set design the, either the lateral or medial side can be longer to accommodate the extended height of such respective side. Still even further, the dynamic swing assist extension mechanism can be with or without a fulcrum and the fulcrum can be either a single or variable fulcrum. In regards to the attachment elements of the knee brace, they can include, but are not limited to, rivets, buckles, hooks, hook and loop material attachments, snap closure elements, screw closure elements, to name just a few.

Equivalent steps for the method as well equivalent elements as both described hereinabove can be substituted for the ones set forth herein to achieve the same results in the same manner and in the same way.

1. A gait corrective and neural rehabilitative orthotic bracing system for use on a patient's leg, foot and ankle, the gait corrective and neural rehabilitative orthotic bracing system comprising:
a) an ankle foot orthosis having a flexible foot plate for positioning below a foot of the patient, at least one upwardly extending strut connected to the foot plate at a lower end, at least one cuff member attached to the at least one upwardly extending strut for positioning around a shin area of the patient's lower leg and at least one strap for securing the ankle foot orthosis to the patient's lower leg; and
b) a knee brace having at least one upwardly extending strut, at least one hinge member intermediately disposed along the at least one hinge member, a means for attaching the knee brace to the patient's lower leg, a means for providing leg swing assistance and a means for increasing patient proprioception.

2. The gait corrective and neural rehabilitative orthotic bracing system of claim 1, wherein the ankle foot orthosis at least one upwardly extending strut has a shape chosen from the group consisting of an anterior, a posterior, a semi-spiral and a spiral shape design.

3. The gait corrective and neural rehabilitative orthotic bracing system of claim 1, wherein the ankle foot orthosis employs a single cuff member positioned at an upper end of the upwardly extending strut distal from the flexible foot plate, the at least one strap attached to the single cuff member.

4. The gait corrective and neural rehabilitative orthotic bracing system of claim 1, wherein the knee brace employs a pair of upwardly extending strut members.

5. The gait corrective and neural rehabilitative orthotic bracing system of claim 4, wherein two hinges are employed, one each disposed on each one of the pair of upwardly extending strut members.

6. The gait corrective and neural rehabilitative orthotic bracing system of claim 4, wherein two hinges are employed, the two hinges chosen for the group consisting of a unicentric hinge and a polycentric hinge.

7. The gait corrective and neural rehabilitative orthotic bracing system of claim 1, wherein the means for attaching the knee brace to the patient's lower leg comprises at least one cuff member of the knee brace.

8. The gait corrective and neural rehabilitative orthotic bracing system of claim 7, wherein the at least one cuff member comprises an upper thigh cuff and a lower shin cuff.

9. The gait corrective and neural rehabilitative orthotic bracing system of claim 1, wherein the means for providing leg swing assistance is a dynamic gait swing assist mechanism mounted on an outer side of the at least one hinge member.

10. The gait corrective and neural rehabilitative orthotic bracing system of claim 1, wherein the means for increasing patient proprioception is chosen form the group consisting of at least one air bladder positioned along an inner side of the at least one upwardly extending strut, a plurality of elastic strapping and a combination of the at least one air bladder positioned along an inner side of the at least one upwardly extending strut and a plurality of elastic strapping.

11. The gait corrective and neural rehabilitative orthotic bracing system of claim 1, wherein the knee brace is applied over top of the ankle foot orthosis.

12. The gait corrective and neural rehabilitative orthotic bracing system of claim 1, wherein the ankle foot orthosis is applied over top of the knee brace.

13. A dual bracing system for correcting abnormal gait in a patient applied to a patient's leg, foot and ankle, the dual bracing system comprising:
a) an ankle foot orthosis having a flexible foot plate for positioning below a foot of the patient, at least one upwardly extending strut connected to the foot plate at a lower end, at least one cuff member attached to the at least one upwardly extending strut for positioning around a shin area of the patient's lower leg and at least one strap for securing the ankle foot orthosis to the patient's lower leg; and
b) a knee brace having at least one upwardly extending strut, at least one hinge member intermediately disposed along the at least one hinge member, a means for attaching the knee brace to the patient's lower leg, a means for providing leg swing assistance and a means for increasing patient proprioception.

14. A method of correcting abnormal gait in a patient, the steps of the method comprising:
a) providing an ankle foot orthosis for attaching to the lower leg of a patient inflicted with abnormal gait, the ankle and foot orthosis having a flexible foot plate for positioning below a foot of the patient, at least one upwardly extending strut connected to the foot plate at a lower end, at least one cuff member attached to the at least one upwardly extending strut for positioning around a shin area of the patient's lower leg and at least one strap for securing the ankle foot orthosis to the patient's lower leg; and
b) providing a knee brace for securing to the knee joint area of the patient's leg, the knee brace having at least one upwardly extending strut, at least one hinge member intermediately disposed along the at least one hinge member, a means for attaching the knee brace to the patient's lower leg, a means for providing leg swing assistance and a means for increasing patient proprioception.

15. The method of claim 14, wherein the ankle foot orthosis is applied to the patient's leg prior to the knee brace such that the knee brace is applied over top of the ankle foot orthosis.

16. The method of claim 14, wherein the knee brace is applied to the patient's leg prior to the ankle foot orthosis knee brace such that the ankle foot orthosis is applied over top of the knee brace.
